(19) United States
(12) Patent Application Publication
(10) Pub. No.: US 2013/0150931 A1
(43) Pub. Date: Jun. 13, 2013

(54) DISTRIBUTED NEURAL STIMULATION ARRAY SYSTEM

(71) Applicants: Daryl R. Kipke, Dexter, MI (US); David Anderson, Ann Arbor, MI (US); Rio J. Vetter, Ypsilanti, MI (US); Jamille Hetke, Brooklyn, MI (US)

(72) Inventors: Daryl R. Kipke, Dexter, MI (US); David Anderson, Ann Arbor, MI (US); Rio J. Vetter, Ypsilanti, MI (US); Jamille Hetke, Brooklyn, MI (US)

(21) Appl. No.: 13/556,638

(22) Filed: Jul. 24, 2012

Related U.S. Application Data
(60) Provisional application No. 61/511,361, filed on Jul. 25, 2011.

(51) Int. Cl. A61N 1/05; H05K 13/00

(52) U.S. Cl. CPC A61N 1/0551; H05K 13/0023
USPC 607/116; 29/592.1; 29/825

(57) ABSTRACT

A device comprises a device carrier having a substantially cylindrical shape and including an array of neural probes. The array of neural probes extend substantially longitudinally from a distal end of the device carrier and include a plurality of central neural probes, wherein a central probe includes at least one stimulating electrode site, and a plurality of peripheral neural probes arranged at a periphery of the plurality of central neural probes, wherein a peripheral neural probe is electrically conducting.

### CLAIM OF PRIORITY

This application claims the benefit of priority under 35 U.S.C. §119(e) of Kipke et al., U.S. Provisional Patent Application Ser. No. 61/511,361, filed Jul. 25, 2011, which is incorporated by reference herein in its entirety.

### TECHNICAL FIELD

This invention relates generally to implantable electrodes, and more specifically to an improved neural stimulation array system in the neural interface field.

### BACKGROUND

Chronic deep brain stimulation (DBS) devices (“brain pacemakers”) are part of a developing approach to the treatment of neurological and psychiatric disorders. Conventional DBS therapy typically involves controllable and selective electrical stimulation of target tissue using a lead with electrodes. The therapeutic effect is largely influenced by the position of the electrodes relative to target tissue, and more specifically, is a function of the spatial and temporal gradients of the stimulation field and of which neuronal structures are influenced by the stimulation charge delivered by the electrodes. However, these conventional DBS devices have limitations as to how the charge is delivered to target tissue. For example, in some applications the ideal stimulation field may include one or multiple selective distributions of charge over a broad volume of tissue, but conventional DBS devices lack the precision or coverage to effectively produce such a stimulation field, without overstimulation or excessive “hot spots” of current that risk tissue damage or because of volume conduction limits in the brain tissue. Thus, the inventors have recognized a need in the electrode lead field to create an improved neural stimulation array.

### BRIEF DESCRIPTION OF THE FIGURES

FIGS. 1A and 1B are schematics showing portions of an example of a distributed neural stimulation array system and a neural probe, consistent with some example embodiments of the invention.

FIGS. 2A, 2B, and 2C are schematics showing an example arrangement of central and peripheral neural probes in an example of a distributed neural stimulation array system, consistent with some example embodiments of the invention.

FIG. 3 shows an example of manufacturing a distributed neural stimulation array system, consistent with some example embodiments of the invention.

### DETAILED DESCRIPTION

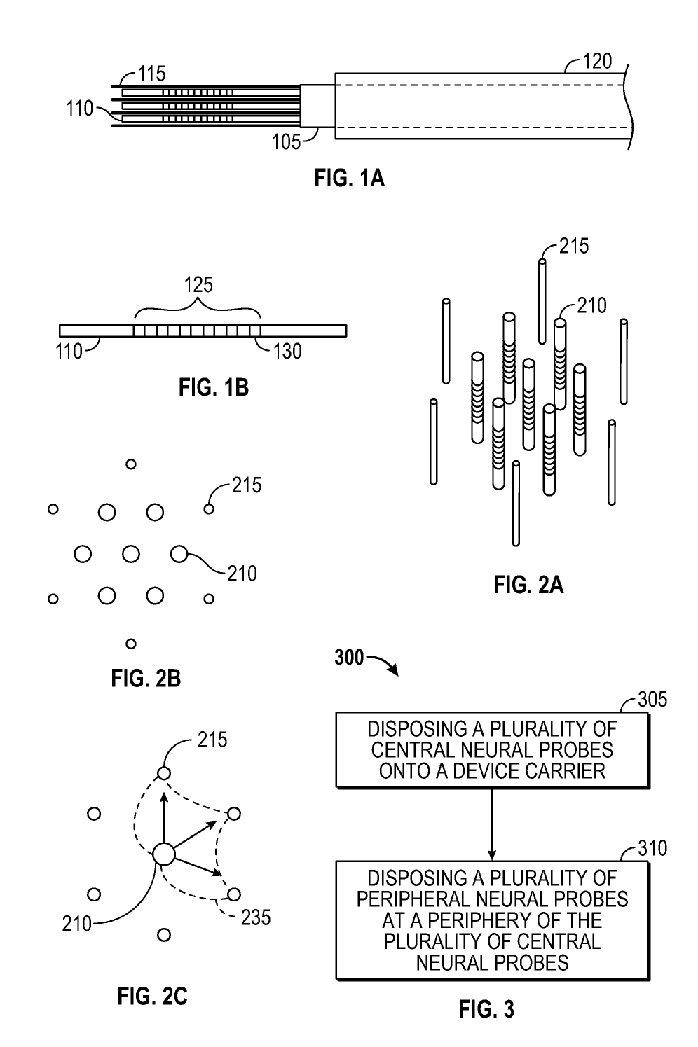

FIGS. 1A and 1B show portions of an example of a distributed neural stimulation array system. The example includes a device carrier 105 and an array of neural probes coupled to the device carrier 105. The device carrier 105 can be substantially cylindrical in shape. Its cross section can be circular, approximately circular, or elliptical. The device carrier 105 can include a proximal end and a distal end. The array of neural probes can extend substantially longitudinally from the distal end of the device carrier 105 as shown in FIG. 1A.

The array of neural probes includes a plurality of central neural probes 110 and a plurality of peripheral neural probes 115 coupled to the device carrier 105. A central neural probe 110 can include at least one stimulating electrode site. In the example of FIG. 1B, a central neural probe 110 includes an electrode array having a plurality of electrode sites. These electrode sites can include one or more stimulating electrode sites and one or more recording electrode sites. In certain examples, an electrode site can be used for both stimulation and recording. The plurality of peripheral neural probes 115 is arranged at a periphery of the plurality of central neural probes 110. The peripheral neural probes 115 can be electrically conducting and can include wire or thin-film electrodes. A peripheral neural probe can be connected (or is connectable) to a current sink circuit, a circuit ground, or a combination of current sinks and ground. In some examples, a peripheral neural probe can be electrically floating.

The distributed neural stimulation array system can be used to provide deep brain stimulation (DBS) or precise electrical brain tissue modulation such as for the treatment of chronic neurological disorders. In various examples, the distributed neural stimulation array system may be implanted in, or near to, any excitable tissue, such as spinal cord tissue, peripheral nerve tissue, muscle tissue, or any suitable tissue to provide a precisely controllable field of stimulation to the tissue. The distributed neural stimulation array system has the potential for customization for specific indications and regions of brain or other tissue. The distributed neural stimulation array system may further include recording electrodes for recording neural activity, as well as channels or other orifices for drug delivery, and/or optical stimulation using optogenetic techniques. Compared to many conventional DBS devices, the distributed neural stimulation array system provides more even and efficient current delivery to surrounding tissue, provides more precise control of the stimulation field or fields, and displaces less tissue, thereby lowering adverse tissue response after implantation.

The device carrier 105 can provide structural support to the central and peripheral neural probes and can include a material of sufficient stiffness to allow the device carrier 105 to function as a delivery shuttle to help position the neural probes into the tissue or other suitable environments. In certain examples, the device carrier 105 can be later removed with the neural probes remaining in position. The device carrier 105 can be substantially cylindrical and flexible, but may alternatively have any suitable shape and/or be rigid or semi-rigid. The device carrier 105 may be made wholly or in part of a resorbable material. The device carrier 105 may be solid (e.g., the neural probes can emanate directly from the distal end of the device carrier 105) or tubular (e.g., the neural probes are carried within a lumen of the device carrier 105 and extend beyond the distal end of the device carrier 105).

In some embodiments, the device carrier 105 is insertable in a guide tube 120 that guides implantation of the array of neural probes into a volume of tissue. When the guide tube 120 is placed near the targeted tissue, the device carrier 105 and neural probes can be advanced from a distal end of the guide tube 120 and selectively placed relative to the targeted tissue. One or both of the device carrier 105 and the guide tube 120 may include one or more guiding elements that position the neural probes in a three-dimensional arrangement. For instance, the guiding element may be a maneuverable guiding element (e.g., a jointed or flexible system of cables or robotics that are controlled by the user to position the neural probes), a biased guiding element (e.g., biased by material choice or structural biasing towards a particular arrangement or position), or other type of guiding element. One or both of the device carrier 105 guide tube 120 may further include a fluidic channel through which a fluidic therapeutic drug or other fluid may be delivered to the tissue.

The plurality of central neural probes 110 can provide electrical stimulation through stimulation electrode sites and/or current steering among the central neural probes and/or peripheral neural probes. As shown in FIG. 2A, the central neural probes 210 can be distributed centrally within the peripheral neural probes 215. Each central neural probe 210 can include an electrode array or an electrode array coupled to a probe carrier. An electrode array includes a plurality of electrode sites and can include at least one stimulation electrode site and at least one recording electrode site. The probe carrier can be a flexible cylindrical or a flexible elliptical probe carrier. The electrode sites of the electrode array can be wrapped circumferentially around the probe carrier. In some variations, the probe carrier can be planar and the electrode array is distributed or arranged on at least one face or edge of the probe carrier. The probe carrier may alternatively have any suitable shape and be rigid or semi-rigid. In another alternative variation, a central neural probe includes an electrode array without a probe carrier.

Similar to the device carrier 105 or FIG. 1A, a probe carrier may include one or both of a guiding element, and a fluidic channel. The probe carrier is preferably less than 1 mm in diameter, and more preferably around 300 μm or less in diameter, which reduces the amount of tissue that each individual neural probe displaces when implanted in tissue; thereby mitigating tissue response to the probe and improving long-term functionality of the probe. The probe carrier may be made of a resorbable material.

As explained previously herein, an electrode array (either with or without a probe carrier) can include at least one stimulation electrode site, and may include one or more recording electrode sites. As shown in FIG. 1B, the electrode array 125 includes ring electrodes 130 distributed along the length of the neural probe. In some examples, the electrode array 125 may include a plurality of electrode sites (e.g. circular or elliptical sites) that are distributed longitudinally along and/or circumferentially around the neural probe. The electrode sites may be electrically coupled to electrical conductors (e.g., insulated interconnects) that terminate at a proximal end to couple to bond pads or to other electrical connections. In some examples, the device carrier 105 is attached to, or integral to, a lead containing a plurality of interconnects electrically coupled to the electrode sites. The electrical conductors may pass through the device carrier 105 to external control systems or other instrumentation.

The electrode sites can be designed (e.g., by one or more of shape, size, material, and position) to provide a combination of selectivity, charge capacity, and efficiency. With more efficient current delivery through the electrode sites (e.g., less voltage required to drive a particular amount of current), current density in the stimulation field may be more evenly distributed across the neural probes and the tissue, which may reduce the overall current density at any particular site. In this manner, the distributed neural stimulation array system is characterized by electrode sites with improved corrosion resistance and the capability of mitigating tissue damage near the electrode sites.

In some examples, the system includes an electronic subsystem coupled to the electrical conductors (e.g., leads electrically coupled to the proximal ends of the electrical conductors). The electronic subsystem may include electronic circuits to provide a stimulation signal to one or more stimulation electrode sites. For instance, each electrode site can be controlled independently based on the therapeutic need, such as by modulating current and/or voltage to the electrode site. Each electrode site may be further controlled individually (e.g., by logic circuits of the electronic subsystem) to stimulate a relatively small volume of surrounding tissue. Individual electrode sites can be referred to as microelectrodes. Multiple electrode sites on a neural probe may be functionally grouped together to form a larger composite macroelectrode site to stimulate a relatively larger volume of tissue. Similarly, any recording electrode site may be operated individually and/or combined in groups by the electronic subsystem to record one or more sensed signals from a neural signal source. In some examples, recording electrode sites are combined to simulate a larger-sized probe. This can be useful to a neurologist who desires to interpret sensed signals as a conventional probe in order to perform conventional analysis. Furthermore, the central neural probes 210 can enable precise current steering to selectively stimulate neural structures or other selective tissue regions. For example, by controlling individual electrode sites and/or collective groups of electrode sites, orientation of stimulation field parameters can be changed. In this way, the system may be used to steer current emitted from the electrode sites around the central and/or peripheral neural probes.

The plurality of peripheral neural probes 215 can function to provide an additional dimension of stimulation field control. As shown in FIGS. 2A and 2B, the peripheral neural probes 215 can be arranged at a periphery of the plurality of central neural probes 210 (e.g., circumferentially arranged in an approximate ring around the central neural probes 210 or a star arrangement around the central neural probes 210). The peripheral neural probes 215 can be regularly spaced apart around the central neural probes 210, but may alternatively be irregularly spaced apart. In a preferred embodiment, at least a portion of the peripheral neural probes 215 are single conductor wire microelectrodes. These wire electrodes may function as current sinks, grounds, and/or floating nodes with no current-carrying capacity. In certain examples, peripheral wire electrodes are configured to carry a current for stimulation and extend the boundaries of the tissue volume that can be controlled without further increasing current densities at the electrode sites on the central neural probes. In certain examples, peripheral wire electrodes can be electrically grounded relative to the central neural probes and the peripheral wire electrodes functions as a “guard” array that limits current (e.g., steered current) from passing outside of a perimeter defined by the peripheral wire electrodes. In other words, a very precisely controlled and modulated stimulation field may be confined within this perimeter or “cage” defined by the grounded (or other-wise connected) peripheral wire electrodes, thereby improving selectivity of tissue stimulation and reducing or preventing inadvertent stimulation of other structures (e.g., tissues of a sensitive nature, non-targeted tissue regions).

FIG. 2C illustrates an example of selective electrical stimulation. A single central neural probe 210 is surrounded by several peripheral neural probes 215. The central neural probe 210 includes several electrode sites while the peripheral neural probes 215 are single conductor probes that can be switched to either ground or floating. The example also shows an electric field (e.g., current field) that results from sourcing current from the central neural probe 210 and switching three of the peripheral neural probes 215 to ground. No simulation gets outside the “cage” formed by the peripheral neural probes 210. The diameter of the probe arrangement is about 2 mm, but no component is more than 0.3 mm.

Similar to the central neural probes 210, the peripheral neural probes 215 may be voltage- or current-controlled independently of each other and controlled independently of the central neural probes 210; thereby providing a high degree of control of neural modulation of a target volume of tissue with the central and peripheral neural probes. In an illustrative example, at any particular time, one portion of the peripheral neural probes 215 may be configured to carry current while another portion of the peripheral neural probes 215 may be electrically grounded. In addition to the wire electrode example, the plurality of peripheral neural probes 215 may additionally and/or alternatively include thin-film neural probes similar to the central neural probes 210. In some variations, peripheral neural probes 215 having highly selectively controllable electrode arrays may enhance current steering of the system.

As shown in FIGS. 2A and 2B, in one non-limiting example, the distributed neural stimulation array system can include seven central, cylindrical neural probes and six peripheral single-channel wire microelectrodes. The central neural probes 210 can include six stimulating electrode rings and can be arranged in an approximately regular hexagonal pattern with six of the central neural probes 210 at the vertices of the hexagon and one of the central neural probes located at the center of the hexagon, such that the seven central neural probes are approximately equally spaced from each other. The six peripheral wire microelectrodes are arranged in a larger approximately regular hexagon concentric with the hexagonally arranged central neural probes. The central and peripheral neural probes can be coupled to a flexible, cylindrical device carrier that is disposed within a guide tube that helps place the carrier in a desired region of tissue.

Other variations may include more or fewer central neural probes, and more or fewer peripheral neural probes. Furthermore, multiple distributed neural stimulation array systems (or multiple groups of central and peripheral neural probes) may be used cooperatively to stimulate, record, or otherwise interact with a volume of tissue.

FIG. 3 shows an example of a method of manufacturing a distributed neural stimulation array system. At block 305, a plurality of central neural probes is disposed onto a substantially cylindrical device carrier. In certain examples, the device carrier includes silicone. The central neural probes extend substantially longitudinally from a distal end of the device carrier. At block 310, a plurality of peripheral neural probes is disposed at a periphery of the plurality of central neural probes. The peripheral neural probes also extend substantially longitudinally from a distal end of the device carrier. Shims can be used when forming the distributed neural stimulation array system to provide the spacing between the probes.

In some examples, the method 300 includes forming an electrode array on one or more of the central neural probes. The electrode array can be formed from a thin-film substrate such as a polymer substrate. The polymer substrate may be parylene, but may additionally and/or alternatively include any suitable material. Layers of materials can be deposited on the substrate and patterned through microfabrication processes such as those used in manufacture of semiconductors or microelectromechanical systems (MEMS). The electrode sites may include a conductive metal such as gold, iridium, or platinum, but may alternatively be any other suitable material.

The electrode array may be coupled to a probe carrier to form a central neural probe. Examples of coupling a thin-film microelectrode array to a carrier are described in Hetke et at, U.S. Pat. No. 7,941,202, filed Oct. 10, 2006, which is incorporated herein by reference in its entirety. Variations of the probe carrier include a substantially cylindrical probe carrier and a planar probe carrier.

The various system and device examples described herein provide precise distribution of charge or current to a tissue target. This results in a precise stimulation field without overbroad stimulation that may adversely affect brain tissue.

### Additional Notes and Examples

Example 1 includes subject matter (such as a device or apparatus) comprising a device carrier having a substantially cylindrical shape and including an array of neural probes. The array of neural probes extending substantially longitudinally from a distal end of the device carrier and includes a plurality of central neural probes and a plurality of peripheral neural probes arranged at a periphery of the plurality of central neural probes. A central probe includes at least one stimulating electrode site and a peripheral neural probe is electrically conducting.

In Example 2, the subject matter of Example 1 optionally includes a central neural probe having an electrode array that includes the at least one stimulating electrode site and at least one recording electrode site.

In Example 3, the subject matter of Example 2 optionally includes an electrode array having a thin-film substrate coupled to a probe carrier.

In Example 4, the subject matter of Example 3 optionally includes a probe carrier that includes resorbable material.

In Example 5, the subject matter of one or any combination of Examples 3 and 4 optionally includes a probe carrier that is substantially cylindrical, and the electrode array optionally includes one or more ring electrodes distributed along a length of the central neural probe.

In Example 6, the subject matter of one or any combination of Example 3 and 4 optionally includes a probe carrier that is planar and wherein the electrode array includes one or more electrodes arranged substantially on a face of the probe carrier.

In Example 7, the subject matter of one or any combination of Examples 3-6 optionally includes at least one of a device carrier and a probe carrier that includes a fluidic channel.

In Example 8, the subject matter of one or any combination of Examples 1-7 optionally includes a device carrier having a longitudinal axis, and the device carrier optionally includes material to provide sufficient stiffness (in an axial direction of the device carrier) for placement of the neural probe array in neural tissue.

In Example 9, the subject matter of one or any combination of Examples 1-8 optionally includes a guide tube configured to receive the device carrier and array of neural probes. The guide tube optionally includes a fluidic channel.

In Example 10, the subject matter of one or any combination of Examples 1-9 optionally includes one or more of the peripheral neural probes that are electrically connectable to a current sink circuit or to a circuit ground.

In Example 11, the subject matter of one or any combination of Examples 1-10 optionally includes a device carrier that includes a plurality of electrical conductors extending from the central and peripheral neural probes to a proximal end of the device carrier.

Example 12 can include subject matter (such as a method, a means for performing acts, or a machine-readable medium including instructions that, when performed by the machine, cause the machine to perform acts), or can optionally be combined with the subject matter of one or any combination of Examples 1-11 to include such subject matter comprising disposing a plurality of central neural probes onto a substantially cylindrical device carrier and disposing a plurality of peripheral neural probes at a periphery of the plurality of central neural probes. A central probe includes a stimulating electrode and a peripheral neural probe is electrically conducting.

In Example 13, the subject matter of Example 12 optionally includes forming an electrode array on one or more of the central neural probes. The forming of the electrode array optionally includes forming at least one stimulating electrode site and at least one recording electrode site formed on a thin-film substrate, and coupling the thin-film substrate to a probe carrier.

In Example 14, the subject matter of Example 13 optionally includes coupling the thin-film substrate to a substantially cylindrical probe carrier, and forming a plurality of circumferential electrodes arranged longitudinally along the central neural probe.

In Example 15, the subject matter of Example 13 optionally includes coupling the thin-film substrate to a substantially planar probe carrier, and forming a plurality of circumferential electrodes arranged substantially on a face of the probe carrier.

In Example 16, the subject matter of one or any combination of Examples 13-15 can optionally include forming a fluidic channel in at least one of the device carrier and the probe carrier.

In Example 17, the subject matter of one or any combination of Examples 12-16 can optionally include electrically connecting one or more of the peripheral neural probes to a current sink circuit or to a circuit ground.

Example 18, the subject matter of one or any combination of Examples 12-17 can optionally include placing the device carrier and array of neural probes in a guide tube that includes a fluidic channel.

In Example 19, the subject matter of one or any combination of Examples 12-18 can optionally include disposing a plurality of electrical conductors in the device carrier that extend from the central and peripheral neural probes to a proximal end of the device carrier.

Example 20 can include subject matter (such as a system), or can optionally be combined with the subject matter of one or any combination of Examples 1-19 to include such subject matter, comprising a device carrier having a substantially cylindrical shape and including an array of neural probes. The plurality of central neural probes extending substantially longitudinally from a distal end of the device carrier and a plurality of peripheral neural probes extending substantially longitudinally from the distal end of the device carrier and arranged at a periphery of the plurality of central neural probes. A central neural probe includes at least one stimulating electrode site and a peripheral neural probe is electrically conducting. The array of neural probes also includes a plurality of interconnects electrically coupled to the array of neural probes. The system also includes an electronic subsystem electrically coupled to the interconnects and configured to provide a stimulation signal to one or more stimulating electrode sites.

In Example 21, the subject matter of Example 20 optionally includes an electronic subsystem configured to provide a stimulation signal to a plurality of stimulating electrode sites and provide independent signal modulation to at least a portion of the plurality of stimulating electrode sites.

In Example 22, the subject matter of one or any combination of Examples 20 and 21 optionally includes at least a portion of the central neural probes including a recording electrode site and the electronic subsystem includes a recording system to record a signal sensed using a recording electrode.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls. 100511 In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A device comprising:
a device carrier having a substantially cylindrical shape and including an array of neural probes, the array of neural probes extending substantially longitudinally from a distal end of the device carrier and including:
a plurality of central neural probes, wherein a central probe includes at least one stimulating electrode site; and
a plurality of peripheral neural probes arranged at a periphery of the plurality of central neural probes, wherein a peripheral neural probe is electrically conducting.

2. The device of claim 1, wherein a central neural probe includes an electrode array including the at least one stimulating electrode site and at least one recording electrode site.

3. The device of claim 2, wherein the electrode array includes a thin-film substrate coupled to a probe carrier.

4. The device of claim 3, wherein the probe carrier includes resorbable material.

5. The device of claim 3, wherein the probe carrier is substantially cylindrical and wherein the electrode array includes one or more ring electrodes distributed along a length of the central neural probe.

6. The device of claim 3, wherein the probe carrier is planar and wherein the electrode array includes one or more electrodes arranged substantially on a face of the probe carrier.

7. The device of claim 3, wherein at least one of the device carrier and the probe carrier includes a fluidic channel.

8. The device of claim 1,
wherein the device carrier includes a longitudinal axis, and
wherein the device carrier includes material to provide sufficient stiffness, in an axial direction of the device carrier, for placement of the neural probe array in neural tissue.

9. The device of claim 1 including a guide tube configured to receive the device carrier and array of neural probes, wherein the guide tube includes a fluidic channel.

10. The device of claim 1, wherein one or more of the peripheral neural probes are electrically connected to a current sink circuit or to a circuit ground.

11. The device of claim 1, wherein the device carrier includes a plurality of electrical conductors extending from the central and peripheral neural probes to a proximal end of the device carrier.

12. A method comprising:
disposing a plurality of central neural probes onto a substantially cylindrical device carrier, wherein the central neural probes extend substantially longitudinally from a distal end of the cylindrical device carrier, and wherein a central probe includes a stimulating electrode; and
disposing a plurality of peripheral neural probes at a periphery of the plurality of central neural probes, wherein the peripheral neural probes extend substantially longitudinally from the distal end of the device carrier, and wherein a peripheral neural probe is electrically conducting.

13. The method of claim 12, including forming an electrode array on one or more of the central neural probes, wherein forming the electrode array includes:
forming at least one stimulating electrode site and at least one recording electrode site formed on a thin-film substrate; and
coupling the thin-film substrate to a probe carrier.

14. The method of claim 13, wherein forming the electrode array includes:
coupling the thin-film substrate to a substantially cylindrical probe carrier; and
forming a plurality of circumferential electrodes arranged longitudinally along the central neural probe.

15. The method of claim 13, wherein forming the electrode array includes:
coupling the thin-film substrate to a substantially planar probe carrier; and
forming a plurality of circumferential electrodes arranged substantially on a face of the probe carrier.

16. The method of claim 13, including forming a fluidic channel in at least one of the device carrier and the probe carrier.

17. The method of claim 12, including electrically connecting one or more of the peripheral neural probes to a current sink circuit or to a circuit ground.

18. The method of claim 12, including placing the device carrier and array of neural probes in a guide tube that includes a fluidic channel.

19. The method of claim 12, including disposing a plurality of electrical conductors in the device carrier that extend from the central and peripheral neural probes to a proximal end of the device carrier.

20. A system comprising:
a device carrier having a substantially cylindrical shape and including an array of neural probes, the array of neural probes including:
a plurality of central neural probes extending substantially longitudinally from a distal end of the device carrier, wherein a central neural probe includes at least one stimulating electrode site;
a plurality of peripheral neural probes extending substantially longitudinally from the distal end of the device carrier and arranged at a periphery of the plurality of central neural probes, wherein a peripheral neural probe is electrically conducting; and
a plurality of interconnects electrically coupled to the array of neural probes; and
an electronic subsystem electrically coupled to the interconnects and configured to provide a stimulation signal to one or more stimulating electrode sites.

21. The system of claim 20, wherein the electronic subsystem is configured to:
provide a stimulation signal to a plurality of stimulating electrode sites; and
provide independent signal modulation to at least a portion of the plurality of stimulating electrode sites.

22. The system of claim 20,
wherein at least a portion of the central neural probes include a recording electrode site, and
wherein the electronic subsystem includes a recording system to record a signal sensed using a recording electrode.
